(19) United States
(12) Patent Application Publication
(10) Pub. No.: US 2010/0125186 A1
(43) Pub. Date: May 20, 2010

(54) MEDICAL DIAGNOSTIC AND COMMUNICATION SYSTEMS

(76) Inventor: Ifeanyi Joemartins Abuachi, Fresno, TX (US)

Correspondence Address: WINSTEAD PC, P.O. BOX 50784, DALLAS, TX, 75201, US

(21) Appl. No.: 12/617,321

(22) Filed: Nov. 12, 2009

Related U.S. Application Data
(60) Provisional application No. 61/114,757, filed on Nov. 14, 2008.

(51) Int. Cl. A61B 5/00; G06Q 50/00

(52) U.S. Cl. 600/301; 705/2

(57) ABSTRACT

Systems and methods for medical diagnosis are described by the present disclosure. In some embodiments, the systems include an input device operable for a patient to enter primary symptoms being experienced, a processing device operable for processing the primary symptoms to form a provisional diagnosis, and an output device operable for providing the provisional diagnosis to the patient. In other embodiments, the systems include an input device operable for a patient to enter medical history and primary symptoms being experienced, a processing device operable for interactively querying the patient based on the medical history and the primary symptoms to determine if additional symptoms are being experienced, a communication device operable for communicating with a physician's office and an output device operable for providing medical advice to the patient. The medical advice is given by the physician's office when the physician's office is open and by the system when the physician's office is closed.

### CROSS-REFERENCE TO RELATED APPLICATIONS

This application claims priority to U.S. provisional patent application 61/114,757, filed Nov. 14, 2008, which is incorporated by reference herein in its entirety.

### STATEMENT REGARDING FEDERALLY SPONSORED RESEARCH

Not applicable

### BACKGROUND

Access to healthcare and physicians is a problem in the United States and throughout the world. Emergency medical care presents a particularly problematic situation. Many times patients experience symptoms in the middle of the night when their physician's office is closed, prompting a trip to the emergency room. Many times such visits are unnecessary and result in needlessly overcrowded emergency rooms and burdensome expenses for patients. Even when patients visit their personal physician for either regular maintenance appointments or in response to symptoms being experienced, patients often feel that their physician has not taken adequate time to allow them to enumerate all of their symptoms or to provide adequate medical history.

In view of the foregoing, it would be beneficial if patients had access to a system that could interactively query their symptoms and provide provisional diagnoses based on their symptoms being experienced. Such a system could significantly lower the number of unnecessary trips to the emergency room by instructing patients when it is most likely acceptable to wait until normal business hours to see their personal physician. Such a system would be further advantageous if it were able to communicate the patient's symptoms, medical information and provisional diagnosis directly to the patient's physician in order to give the physician a complete picture of the patient's presentation before the physician meets with the patient. Systems described by the present disclosure meet these unmet needs by collecting medical information and providing medical advice when a physician is not otherwise available to a patient.

### SUMMARY

In various embodiments, the present disclosure describes medical diagnostic systems including an input device operable for a patient to enter primary symptoms being experienced, a processing device operable for processing the primary symptoms to form a provisional diagnosis, and an output device operable for providing a provisional diagnosis to the patient.

In other various embodiments, medical diagnostic systems of the present disclosure include an input device operable for a patient to enter medical history and primary symptoms being experienced, a processing device operable for interactively querying the patient based on the medical history and the primary symptoms to determine if additional symptoms are being experienced, a communication device operable for communicating with a physician's office and an output device operable for providing medical advice to the patient. The medical history, primary symptoms and the additional symptoms are automatically transmitted to the physician's office with the communication device.

In still other various embodiments, methods for diagnosing a patient are described herein. The methods include providing a medical diagnostic system, inputting the patient's primary symptoms and medical history into the medical diagnostic system and receiving a provisional diagnosis from the medical diagnostic system.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

For a more complete understanding of the present disclosure, and the advantages thereof, reference is now made to the following descriptions to be taken in conjunction with the accompanying drawings describing specific embodiments of the disclosure, wherein:

FIG. 1 presents an illustrative flowchart showing an embodiment of the present disclosure in which a patient receives a diagnosis from the system; and

FIG. 2 presents an illustrative flowchart showing an embodiment of the present disclosure in which a patient receives a diagnosis from either from a physician's office or from the system depending on whether the physician's office is open or closed.

### DETAILED DESCRIPTION

In the following description, certain details are set forth such as specific quantities, sizes, etc. so as to provide a thorough understanding of the present embodiments disclosed herein. However, it will be evident to those of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing particular embodiments of the disclosure and are not intended to be limiting thereto. Drawings are not necessarily to scale.

While most of the terms used herein will be recognizable to those of ordinary skill in the art, it should be understood, however, that when not explicitly defined, terms should be interpreted as adopting a meaning presently accepted by those of ordinary skill in the art. In cases where the construction of a term would render it meaningless or essentially meaningless, the definition should be taken from Webster's Dictionary, 3rd Edition, 2009. Definitions and/or interpretations should not be incorporated from other patent applications, patents, or publications, related or not, unless specifically stated in this specification or if the incorporation is necessary for maintaining validity.

As used herein, the term “adrenaline buzz” will be used to describe a condition resulting from anxiety experienced by a patient as a result of newly experienced medical symptoms, particularly when the cause of those symptoms is unknown.

As used herein, the term “clarksman” or “clarksmanship” will be used to describe a system or process whereby medical history from a patient is gathered and reviewed and the patient is further interactively queried to determine associated factors that may have contributed to symptoms being experienced or to determine if additional symptoms are also being experienced. For example, in a patient complaining of shortness of breath and cough of protracted duration, the clarksman might elicit a history of smoking. Approximately 85% of all medical diagnoses can be determined by a physician's consideration of a patient's comprehensive medical history and symptoms through clarksmanship. However, a typical physician has limited time in which to gather and consider a patient's medical history during an office visit.

As used herein, the term “medical history” will be used to refer to accumulated general information on a patient including, for example, family (genetic) history, social history, habits and prior medical information that can help a diagnosis to be reached.

There are a number of reasons why patients are reluctant to visit their personal physician. These reasons include, for example: 1) personality type, 2) age, 3) sex, 4) financial situation, 5) education, and 6) available time. However, in some instances persons are generally compelled to seek medical attention, regardless of their background. These instances include severe pain, injury or emergency situation. In non-emergency situations, persons are generally compelled to seek medical advice when they experience strange symptoms that create general anxiety and panic. The systems and methods of the present disclosure seek, amongst other things, to mitigate the adrenaline buzz experienced by patients through providing medical advice (for example, a provisional diagnosis or directions to seek emergency medical attention) when the patient cannot or will not be able to see their personal physician. The systems and methods of the present disclosure advantageously allow patients to provide medical information to the system in a comfortable place, such as their own homes, and the patients, in turn, receive a provisional diagnosis based on their symptoms being experienced until they are able to visit their personal physician for a confirming diagnosis. Furthermore, many patients become overwhelmed upon visiting a physician's office, and the anxiety they experience subsequently alters or exacerbates their prevailing symptoms and vital signs such as, for example, blood pressure, pulse rate and respiratory rate. As a result of this anxiety, some patients may even forget to tell their physician about key symptoms that could have been useful in making an accurate diagnosis. The systems and methods of the present disclosure overcome these limitations of conventional doctor-patient interactions.

Further, in some embodiments, the systems and methods of the present disclosure allow symptoms and medical information provided by the patient to be directly transmitted to a physician so that the physician has direct access to a complete and current medical profile of the patient. By directly communicating these data to the physician, the physician is advantageously allowed to review the patient's presentation and make tentative diagnoses and decisions before direct or indirect consultation with the patient even occurs. In some embodiments, the physician's diagnosis may even be aided by a provisional diagnosis made by the system. Such communication of the patient's medical information and symptoms may make more efficient use of the physician's time, thereby improving the efficiency of the healthcare system as a whole. Still further, patient outcomes may be improved by the systems and methods of the present disclosure by reducing the possibility of misdiagnosis and more effectively managing complications. By improving patient outcomes and improving physician efficiency, corresponding strain on the insurance industry will be mitigated.

In general, the systems and methods of the present disclosure are distinguished from and advantageous over online medical search engines that are conventional in the art, because the systems and methods of the present disclosure use a symptoms-based approach for diagnosis, rather than starting from a diagnosis from which patients attempt to match their symptoms. Further, the systems and methods of the present disclosure interactively query the patient based on their medical history and primary symptoms being experienced to solicit further symptom information prior to generating a provisional diagnosis. Still further, in some embodiments, the systems and methods of the present disclosure offer capabilities to communicate medical history and symptom information directly to a physician's office to keep the patient's physician fully apprised of the patient's condition without the patient actually having to visit the physician's office.

In various embodiments, the present disclosure describes medical diagnostic systems including an input device operable for a patient to enter primary symptoms being experienced, a processing device operable for processing the primary symptoms to form a provisional diagnosis, and an output device operable for providing a provisional diagnosis to the patient. In various embodiments, the input device is further operable for soliciting a medical history from the patient.

In some embodiments, the processing device returns a provisional diagnosis after evaluating the primary symptoms. However, in other various embodiments, the symptoms may not be consistent with a particular diagnosis, or the number of possible diagnoses may be too large to be clinically useful. In these cases, the systems and methods of the present disclosure are able to solicit more information from the patient regarding their symptoms.

In various embodiments, the processing device is further operable for interactively querying the patient to determine if additional symptoms are being experienced. In some embodiments, interactively querying takes place by a clinical clarksmanship protocol based upon primary symptoms and medical history entered by the patient. The clinical clarksmanship protocol is based upon the symptom-based interview methods that physicians use in a clinical setting when evaluating a patient. In various embodiments, the processing device includes, for example, a computer and associated software. Such software may utilize a database of medical information and a decision tree structure based on clinical clarksmanship to filter the patient's medical information in order to arrive at a provisional diagnosis.

Various forms of input devices are suitable for use in the systems and methods of the present disclosure. In some embodiments, input devices can include, for example a computer with a keyboard entry or touchscreen entry or a telephone computer data entry system. In other embodiments, multiple input devices can be used by the patient to input symptoms and medical complaints and history into the system. For example, the patient may enter medical history on a computer when a patient's user account is first set up and then use a telephone computer data entry system thereafter to input symptoms and respond to queries posed by the system.

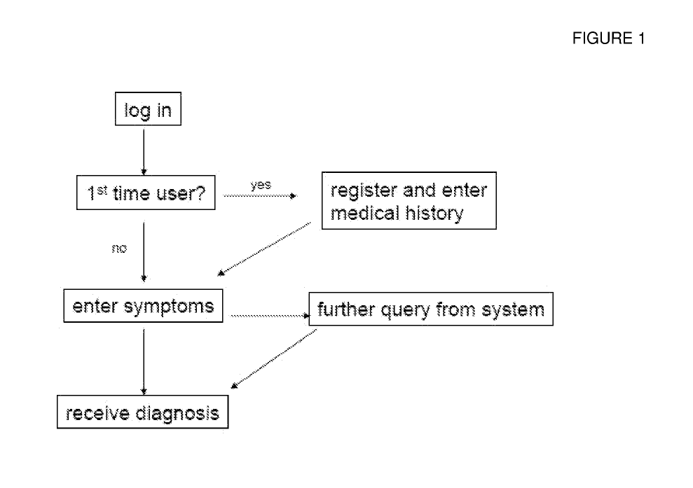

FIG. 1 presents an illustrative flowchart showing an embodiment of the present disclosure in which a patient receives a diagnosis from the system. As shown in FIG. 1, upon entering the system, the patient logs in to his personal account. If the patient is a first time user, he enters medical history information to be used by the system in making a diagnosis. If the patient is a returning user, he directly enters his symptoms using the input device. After entering the symptoms, the system may then either return a provisional diagnosis or further query the patient for additional symptom information. Upon obtaining additional symptom information, the system returns a provisional diagnosis to the patient and provides recommended medical advice. For example, the patient may be advised to proceed immediately to the emergency room or wait until normal business hours to see their personal physician. The provisional diagnosis may be a single diagnosis, or it may be a listing of possible diagnoses, listed in order of increasing likelihood.

In general, first time users of the system log on and create a user ID and password/PIN to be used for accessing the system. First time users of the system are also prompted to enter medical history and appropriate personal information. Illustrative information to be entered includes, for example, name, sex, educational history, age, height, weight, occupation, marital status (optional), prior medical history and surgeries, family history of disease [e.g., diabetes, hypertension, heart disease, lung disease, liver disease (e.g., jaundice, cirrhosis, enlargement), cancer, bone disease, joint disease (e.g., arthritis), high cholesterol, glaucoma, psychiatric disorder, depression, anxiety/panic attacks, suicidal thoughts, gout, allergies, gall stones, kidney stones and kidney disease. Social habits are also entered at this time including, for example, alcohol use, smoking, sexual practices (including number of partners), illegal drug use (e.g., marijuana, cocaine, heroin), oral tobacco use and coffee consumption. Medication history is also entered at this time including, for example, pain pills, antibiotics, blood thinners, chemotherapy drugs, antifungals, antiretrovirals, antivirals, and thyroid drugs. Medical history and family history are also entered at this time and are saved by the system for the next time the patient accesses the system. Female patients are also asked to enter the date of their last menstrual period, age of menarche, pregnancy history, abortion history (both spontaneous and medical), miscarriage history, number of live births, number of children, date of last child birth, breast feeding history, and family history of gynecological and/or breast disease. Finally, the patient is prompted to enter the symptoms currently being experienced.

In some embodiments, the systems of the present disclosure further include a communication device operable for communicating with a physician's office. In further embodiments, the systems further include a communication device operable for a physician's office to communicate with the patient. By using the communication device, information entered into the system by the patient (e.g. primary symptoms, additional symptoms and medical history) may be transmitted to the physician's office for review by a physician. The provisional diagnosis by the system may also be transmitted to the physician's office for the physician's reference. Likewise, the physician may utilize his communication device to proactively communicate with the patient and provide medical advice. The physician's communication device is also operable to receive information that the patient entered into the system.

In some embodiments, the processing device and the communication device operable for communicating with the physician's office may be the same. For example, the processing device and the communication device may both be a computer and associated software. In this embodiment, primary symptoms, additional symptoms, medical history and provisional diagnosis may be transmitted to a physician's office in electronic form such as, for example, an electronic mail or an electronic chart format. However, the processing device and the communication device may be different in other embodiments. For example, the processing device may be a computer and associated software and the communication device may be an automated telephone message transmitting information to or from a physician's office. When a telephone is the communication device, the system may prompt the patient to enter symptoms or information on health status using numbers on the telephone keypad. Alternatively, the system may include voice recognition software for inputting patient information, either on the computer or telephone.

In some embodiments, the communication device for communicating with the physician's office utilizes the Internet for transmitting patient information. In such embodiments, the patient input device for the system may be a web browser interface. In other embodiments, the communication device for communicating with the physician's office may be a computer running specialized software that is not web-based. The computer may transmit the information to the physician's office by various routes, including, for example, wirelessly or over the phone lines through a modem. In some embodiments, the system may include an instrument run by a computer, wherein the input device is just a few buttons, rather than a keyboard interface of a traditional computer.

Each patient will have his own device housing the systems of the present disclosure and having a communication device for communicating with his physician's office. The physician is not required to have multiple devices for each patient, however. In various embodiments, the physician may use a single device to evaluate all patients that log into his practice. For example, the physician may have a single communication device that can be used to communicate and receive information from all of his patients.

In some embodiments, the physician's communication device may communicate with the communication device of other physicians. By communicating with other physicians' communication devices, all physicians that a patient visits will have access to the patient's comprehensive medical history. Such a communication system between physicians could be used to prohibit patients (particularly Medicare patients) from visiting two different physicians within a given period of time for the same indication. Accordingly, financial burden on the Medicare system would be lessened in this embodiment.

In further embodiments, medical laboratories may log on to the physician's system and communicate test results ordered by the physician. For example, such test results may be directly downloaded into a patient's user account. In this way, the physician may have at his disposal the most complete and up-to-date view possible of the patient's medical profile.

In some embodiments, the input device further includes a data collection interface operable for collecting at least one vital sign for entry into the system. Vital signs include, for example, blood pressure, pulse rate, respiratory rate and blood glucose levels. Collected vital sign information may be stored by the system and then be automatically transmitted to the physician's office according to embodiments of the present disclosure.

In some embodiments, the input device further includes a graphical interface for inputting primary symptoms. In some instances, a patient may be best able to describe their symptoms in terms of a representative picture rather than by a written description. In these instances, the system includes a database of selectable pictures that the patient may use for supplying symptom information.

In certain embodiments, the graphical interface includes a database of information for helping a patient select a dermatological condition being experienced. Symptoms such as, for example, itching, burning, scaly, may be able to be described by the patient. However, the patient may not be able to describe diagnostic features such as, for example, macules, papules, vesicles, nodules, hyperpigmentation, hyperkeratosis, and lichenification to help secure the proper diagnosis. The system can present pictures of these conditions to the patient in order to aid the patient in describing their symptoms.

In other various embodiments of the present disclosure, medical diagnostic systems include an input device operable for a patient to enter medical history and primary symptoms being experienced, a processing device operable for interactively querying the patient based on the medical history and the primary symptoms to determine if additional symptoms are being experienced, a communication device operable for communicating with a physician's office and an output device operable for providing medical advice to the patient. The medical history, primary symptoms and the additional symptoms are transmitted to the physician's office with the communication device.

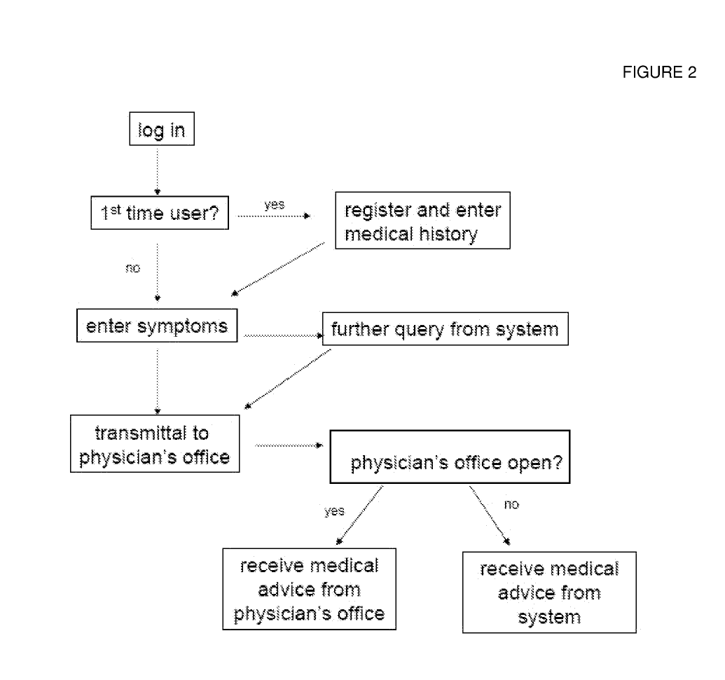

In some embodiments, the medical advice is provided by the physician's office when the physician's office is open. However, in other embodiments, the medical advice is provided by the processing device when the physician's office is closed. FIG. 2 presents an illustrative flowchart showing an embodiment of the present disclosure in which a patient receives a diagnosis from either from a physician's office or from the system depending on whether the physician's office is open or closed. In these embodiments, the system serves as a remote waiting room for the patient by providing medical information to the patient's physician without the patient actually having to visit the physician's office and as a surrogate physician when the patient's physician is otherwise unavailable. When the physician's office is closed, the processing device of the system processes the medical history, primary symptoms and additional symptoms to form a provisional diagnosis.

In some embodiments of the present disclosure, the system can further review medications being taken by the patient and medical conditions that are part of the patient's medical history. The system may then analyze these conditions to interactively query the patient as to why they might be accessing the system. For example, in a diabetic patient, the system may ask the patient if he has forgotten to take insulin, feels tired, hungry or has blurred vision. Depending on how the patient responds to these inquires, further clarksmanship may be performed by the system to determine the source of the patient's symptoms. For example, if the patient responds yes to any of the inquires, the system may request that patient check his blood glucose level. In the presence of abnormal glucose levels, the system may again query whether the patient took their medications on schedule, and, if so, the patient is advised to proceed to the emergency room. Otherwise, the patient is advised to take their medication and re-evaluate their medical condition after the medication has had time to take effect.

Embodiments of the present disclosure are also particularly advantageous for the elderly or persons living in remote settings who do not have routine access to a physician. For example, in embodiments of the systems of the present disclosure having a communication device for communicating with a physician's office, the patient may be able to receive direct instructions from their personal physician upon the physician's review of the patient's symptom and medical information collected by the system. As an illustrative example, elderly patients may be particularly prone to be hypertensive, diabetic or both, and such patients may be at risk for complications such as, for example, stroke, diabetic coma and renal failure. The systems of the present disclosure allows such at-risk patients to monitor their symptoms and automatically communicate them to a physician's office, which can allow medical intervention to be initiated prior to any complications becoming severe. For example, the system may be able to establish when the patient has not conducted routine monitoring of their blood glucose or blood pressure levels or if the patient has forgotten to take their blood pressure medication. Further, a physician's office may be able to intervene on behalf of the patient or request that they come in for a visit to the physician's office.

The systems of the present disclosure may also include login information for a primary user and subaccounts for a dependent, such as an elderly parent or child. For example, a patient may log on to the system and input symptom and medical history information for their ill child. In addition to medical history information described hereinabove, a child's medical information may also include, for example, age, weight (for accurate dosing of medications), pregnancy history to determine if the child's symptoms may have been a result of events that occurred during pregnancy or during delivery, jaundice, feeding and surgical histories, and immunization history. Many parents are exceedingly protective of their children and seek a physician's input at even the slightest sign of illness in their child. The systems of the present disclosure may mitigate the adrenaline buzz experienced by parents of an ill child upon learning of their child's illness by providing medical advice to the parents and instructing them when symptoms being experienced should be considered an emergency. Accordingly, unnecessary trips to the emergency room by the child may be minimized through use of the systems of the present disclosure.

In another embodiment of the medical diagnostic systems of the present disclosure, the subaccount information may be for a pet. For example, the medical history and symptom information and clarksman procedures described herein may be modified for veterinary diagnosis of a variety of animals. The systems described herein may be particularly applicable for maintaining animal health, since animals have no direct means for communicating with a veterinarian. However, when a pet owner inputs the pet's symptoms, behavior and medical history into the system, the clarksman may be able to provide a provisional diagnosis of the pet's condition. Appropriate referral to the pet's veterinarian may then be recommended by the system.

In other various embodiments, methods for diagnosing a patient are described herein. The methods including providing a medical diagnostic system according to the embodiments described herein, inputting the patient's primary symptoms and medical history into the medical diagnostic system and receiving a provisional diagnosis from the medical diagnostic system. In some embodiments, the methods further include querying the patient for additional symptom information using the medical diagnostic system. In some embodiments, the methods further include communicating the patient's primary symptoms, medical history and provisional diagnosis to a physician's office using the medical diagnostic system. In some embodiments, the patient's additional symptom information is also communicated to the physician's office by the medical diagnostic system.

### Experimental Examples

The following examples are provided to more fully illustrate some of the embodiments disclosed hereinabove. It should be appreciated by those of ordinary skill in the art that the techniques disclosed in the examples that follow represent techniques that constitute illustrative modes for practice of the disclosure. Those of ordinary skill in the art should, in light of the present disclosure, appreciate that many changes can be made in the specific embodiments that are disclosed and still obtain a like or similar result without departing from the spirit and scope of the disclosure.

In the case studies that follow, representative patient presentations are described, which are intended to demonstrate particular aspects of the medical diagnostic systems of the present disclosure. The case histories that follow are merely illustrative and should not be considered as limiting of the scope of the embodiments of the present disclosure. It should be noted that the case histories are illustrative and not based on medical data from an actual patient.

Case Study 1: Nose Bleed. Usually, a patient recognizes when he or she has dizziness, headaches, fever with chills, and bleeding from nose, but will not be able to relate these signs and symptoms to a specific result. The mere sight of blood from the nose is major cause for concern. However, the blood could be due to a life-threatening condition such as, for example, high blood pressure or tumor, or non-life-threatening rhinitis, which is an inflammation of the mucus lining of the nose. Further, sinusitis could explain the fever and chills in combination with a nosebleed. The medical diagnostic systems of the present disclosure will help a patient assess these symptoms to determine if immediate medical attention is needed.

In the present case study, a 48-year old patient observes blood dripping out of his nostrils. He logs on to the system and is then prompted to type in his symptoms, namely, nose bleed, headache, dizziness and fever/chills. The system will derive a number of tentative diagnoses, namely, hypertension, sinusitis, medication-induced bleeding, blood dyscraziers and neoplasm. The tentative diagnoses will be made in order of frequency of occurrence.

Hypertension is a major cause of nose bleeds, because the high pressure exerts a force on the walls of the tiny vessels in the nose, leading to their rupture and bleeding. Some medications such as, for example, blood thinners can induce nose bleeds. There are also people with genetic abnormalities of red blood cells and/or platelets who can present with nose bleeds. Sinusitis can cause nose bleed following an inflammatory phase in the nasal mucosa, bogginess of the mucosa and blood vessels, with high susceptibility to rupture. Neoplastic conditions of the nose/pharynx e.g. the nasopharyngeal carcinoma, or of the brain may present with nose bleed from local invasion and rupture of nearby blood vessels.

Because hypertension is a common factor that causes nose bleeds, the system may prompt patient to check his blood pressure. If the patient returns a pressure reading of, for example, 180/120 mmHg or more, the likelihood of hypertension will guide the system to prompt the patient to proceed to the emergency room. In absence of a blood pressure measurement apparatus, patient will receive the provisional diagnoses and then be advised to proceed to the emergency room. If blood pressure is normal, hypertension is apparently ruled out, and the clarksman will be activated to check the patient's medical history for prior history of hypertension, family history of hypertension or a history of taking blood pressure medications. If any of these conditions are established, the clarksman will suspect an error in the blood pressure check and prompt a recheck or trip to the emergency room.

If the clarksman fails to establish prior history of hypertension, family history of hypertension, or no history of taking blood pressure medications, the tentative diagnoses will then focus on allergic rhinitis, sinusitis (considering the associated chills and fever) and possible new incidence of hypertension. In this instance, the patient will be advised to proceed to the emergency room or to see his personal physician.

If the clarksman elicits a history of bleeding disorders or a patient on drugs such as, for example, aspirin or blood thinners, then blood dyscraziers will be likely. Accordingly, the patient will be advised to proceed to the emergency room or to see his personal physician immediately.

If the clarksman fails to establish positive history in a young male or female, then the likely causes for the nose bleed will then be sinusitis or allergic rhinitis. Under these conditions, the patient will still be advised to proceed to the emergency room or to follow up with their personal physician, but fears from an adrenaline buzz will be allayed.

Case Study 2: Vaginal Bleeding and Nausea. In the present case, the patient is a 26 year old female. She logs on to the system and is then prompted to type in her symptoms, namely, abdominal pain, vaginal bleeding, nausea/vomiting and feeling of faintness or weakness. The system will derive a number of provisional diagnoses, namely, pregnancy, ectopic pregnancy, dysmenorrhea, cervicitis and endometritis. To narrow down the provisional diagnoses, the system will activate the clarksman to determine history of the patient's last menstrual period (LMP) and past history of sexually transmitted disease (STD).

If LMP is more than three weeks previous, pregnancy (most likely ectopic or threatening abortion) will be suspected, and the patient will be advised to proceed to the emergency room (ER). If the LMP is less than three weeks previous, but the clarksman elicits a history of vaginal discharge, painful voiding of urine, and/or painful intercourse, pelvic inflammatory disease (PID), cervicitis, endometritis or urinary tract infection will be suspected.

If there is prior history of STD, the clarksman will request that the patient check their blood pressure. In the instance of a prior history of STD plus low blood pressure, a ruptured tubal pregnancy will be the most likely diagnosis, and the patient will be advised to proceed to the emergency room immediately. If the clarksman establishes additional symptoms of fainting spells, generalized weakness and syncopy, a provisional diagnosis of ectopic pregnancy will likely be returned.

If there is no history of STD and LMP is more than three weeks previous with no vaginal discharge, no painful voiding of urine (dysuria), normal blood pressure and negative pregnancy test, the system will return a most likely diagnosis of painful menstrual period (dysmenorrhea). The system will also try to eliminate other causes of vaginal bleeding in a woman of that age group by eliciting more symptoms or history from the patient via the clarksman. For example, the clarksman may diagnose cervicitis (i.e. inflammation of the cervix), vaginal tear or trauma, or endometritis (inflammation of the internal lining of the uterus).

Case Study 3: Bloody Urine. In this case study, the patient is a 26 year old male factory worker. He observes blood in his urine when he wakes up in the middle of the night to void urine. He had back pain for one day and had taken a pain pill to relieve his pain. Upon logging into the system, he enters symptoms of blood in urine and back pain. Based on these two symptoms alone, a number of tentative diagnoses are possible. For example, urinary tract infection, urinary calculus (i.e., kidney stone), urethral trauma, neoplasm (cancer) of the renal system, and side effects of certain drugs are possible diagnoses.

To narrow down the number of possible diagnoses, the system will analyze the medical history to determine if any information is present that can help narrow down the possible diagnoses. For example, the system may determine that there is a family history of kidney stones. Accordingly, the clarksman will solicit more information from the patient centering around this possible cause of the bloody urine. For example, the clarksman will solicit more information about the bloody urine itself (was the whole stream discolored red, or was the first or terminal part of the stream discolored). When the whole stream is discolored, kidney parenchyma is suspected. Discoloration at the start of the stream is indicative of trauma in the urethra, whereas discoloration at the end of the stream is indicative of a blood source in the bladder. Any of these conditions can result from kidney stones, depending on where the stone is lodged. The blood could also have resulted from the pain medication taken. The system will withhold making a provisional diagnosis until further information on the back pain has been gathered.

Turning to the back pain the clarksman will solicit further information on the pain such as, for example, whether it was of sudden and insidious onset, whether it travels or is localized, what makes the pain worse and what relieves the pain. In the instance of a kidney stone, it would be expected that the patient would describe the onset of pain as sudden and travelling up and down his back to the groin area.

In querying the patient, the system will provide the patient a menu where further information such as that detailed above can be entered. For example, the patient may click a check box next to each piece of additional information relevant to the symptoms being experienced. Assuming the patient enters a family history of kidney stones, sudden onset of pain that waxes and wanes and travels down to the groin area, no fever and pain relieved by a pain pill, the system will return a provisional diagnosis of a renal calculus (resulting in the back pain) and a drug-induced hematuria (resulting in the blood in the urine).

From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure, which is defined in the following claims.

What is claimed is the following:

1. A medical diagnostic system comprising:
an input device operable for a patient to enter primary symptoms being experienced;
a processing device operable for processing the primary symptoms to form a provisional diagnosis; and
an output device operable for providing the provisional diagnosis to the patient.

2. The medical diagnostic system of claim 1, wherein the input device is further operable for soliciting a medical history from the patient.

3. The medical diagnostic system of claim 2, wherein the processing device is further operable for interactively querying the patient to determine if additional symptoms are being experienced.

4. The medical diagnostic system of claim 3, wherein interactively querying the patient comprises a clinical clarksmanship protocol based upon the primary symptoms and the medical history.

5. The medical diagnostic system of claim 3, further comprising:
a first communication device operable for communicating with a physician's office;
wherein the primary symptoms, the additional symptoms, the medical history and the provisional diagnosis are automatically transmitted to the physician's office.

6. The medical diagnostic system of claim 5, wherein the first communication device is selected from the group consisting of a telephone and a computer.

7. The medical diagnostic system of claim 5, further comprising:
a second communication device operable for the physician's office to communicate with the patient;
wherein the second communication device is further operable to receive the primary symptoms, the additional symptoms, the medical history and the provisional diagnosis from the first communication device.

8. The medical diagnostic system of claim 1, further comprising:
a first communication device operable for communicating with a physician's office;
wherein the primary symptoms and the provisional diagnosis are automatically transmitted to the physician's office.

9. The medical diagnostic system of claim 8, further comprising:
a second communication device operable for the physician's office to communicate with the patient;
wherein the second communication device is further operable to receive the primary symptoms and the provisional diagnosis from the first communication device.

10. The medical diagnostic system of claim 1, wherein the input device further comprises a graphical interface for inputting the primary symptoms.

11. The medical diagnostic system of claim 1, wherein the input device further comprises a data collection interface operable for collecting at least one vital sign for entry into the system;
wherein the at least one vital sign is selected from the group consisting of blood pressure, pulse rate, respiratory rate, blood glucose level and combinations thereof.

12. A medical diagnostic system comprising:
an input device operable for a patient to enter medical history and primary symptoms being experienced;
a processing device operable for interactively querying the patient based on the medical history and the primary symptoms to determine if additional symptoms are being experienced;
a communication device operable for communicating with a physician's office;
wherein the medical history, the primary symptoms and the additional symptoms are automatically transmitted to the physician's office by the communication device; and
an output device operable for providing medical advice to the patient.

13. The medical diagnostic system of claim 12, wherein the medical advice is provided by the physician's office when the physician's office is open.

14. The medical diagnostic system of claim 12, wherein the medical advice is provided by the processing device when the physician's office is closed.

15. The medical diagnostic system of claim 12, wherein the processing device is further operable for processing the medical history, primary symptoms and additional symptoms to form a provisional diagnosis.

16. The medical diagnostic system of claim 12, wherein the communication device is selected from the group consisting of a telephone and a computer.

17. The medical diagnostic system of claim 12, wherein the input device further comprises a graphical interface for inputting the primary symptoms.

18. The medical diagnostic system of claim 12, wherein the input device further comprises a data collection interface operable for collecting at least one vital sign for entry into the system;
wherein the at least one vital sign is selected from the group consisting of blood pressure, pulse rate, respiratory rate, blood glucose level and combinations thereof.

19. A method for diagnosing a patient, said method comprising:
providing the medical diagnostic system of claim 1;
inputting the patient's primary symptoms and medical history into the medical diagnostic system; and
receiving a provisional diagnosis from the medical diagnostic system.

20. The method of claim 19, further comprising:
communicating the patient's primary symptoms, medical history and provisional diagnosis to a physician's office using the medical diagnostic system.
